(19) United States
(12) Patent Application Publication
(10) Pub. No.: US 2024/0225755 A9
(43) Pub. Date: Jul. 11, 2024

(54) CONTROL CONSOLE DISPLAY AND IDENTIFICATION METHOD FOR SURGICAL TOOLS

(71) Applicant: Arthrex, Inc., Naples, FL (US)

(72) Inventors: Phil O'Quinn, Naples, FL (US); Jonathan Knopf, Naples, FL (US); Joshua Buckman, Naples, FL (US); Robert Breckner, Reno, NV (US); Edwin Floyd, Naples, FL (US)

(73) Assignee: Arthrex, Inc, Naples, FL (US)

(21) Appl. No.: 18/381,862

(22) Filed: Oct. 19, 2023

Related U.S. Application Data
(60) Provisional application No. 63/418,799, filed on Oct. 24, 2022.

(51) Int. Cl. A61B 34/00; A61B 90/94; A61B 90/98

(52) U.S. Cl. CPC A61B 34/25; A61B 90/94; A61B 90/98

(57) ABSTRACT

A method for controlling a surgical device includes accessing identification information from an identification device of at least one interchangeable accessory. The method further includes configuring an operating configuration of at least one surgical device in response to the identification information. The control instruction is communicated for an actuator of the at least one surgical device via a first port of a plurality of ports of a connection interface. The first port of the connection interface is distinguished from the plurality of ports of the connection interface by displaying an identification symbol identifying the interchangeable accessory on a display screen.

### CROSS-REFERENCE TO RELATED APPLICATIONS

This application claims priority under 35 U.S.C. § 119(e) and the benefit of U.S. Provisional Application No. 63/418,799 entitled CONTROL CONSOLE DISPLAY AND IDENTIFICATION METHOD for surgical Tools, filed on Oct. 24, 2022, by O'Quinn et al., the entire disclosure of which is incorporated herein by reference.

### BACKGROUND

The present disclosure generally relates to a system and method for controlling surgical tools. The operation of the system may provide for intuitive control that may otherwise be challenging or complicated, particularly when implementing modular tool assemblies with interchangeable components.

### SUMMARY

The disclosure provides for systems and methods that may relate to a comprehensive control environment for operating surgical tools with interchangeable components or accessories. The operation of such systems can present difficulties due to the combined requirements of allowing broad variations for operation, while providing intuitive operation. In some cases, control systems may include colored indicator lights that may assist users in identifying connected surgical tools. However, such solutions may only provide the limited benefit identifying the connectivity of devices and may not be effective in assisting an operator in distinguishing among surgical tools with a variety of configurable functions. Accordingly, the disclosure may provide for improved detection and operation of surgical tools with interchangeable accessories by detecting and presenting symbols and information to intuitively identify the connected devices.

In various implementations, the disclosed systems and methods may provide for the operation of a control console configured to identify and control a plurality of connected surgical tools. As discussed in various examples, the surgical tools may correspond to handpieces or hand-operated power tools, as well as other surgical or medical devices. In order to configure the operation of the control console to suit the diverse operating requirements, the control console may distinguish among various tool configurations of the surgical tools as well as identify operating information for various interchangeable accessories. Accordingly, the control console and corresponding systems may provide for the identification and control of a wide variety of surgical tools, each of which may be compatible with various interchangeable accessories.

The wide range of configurations among the surgical tools and interchangeable accessories may require the control console to adjust among a similarly wide variety of control configurations. In addition to accessing and controlling the configurations for each of the tools and accessories, the system may further provide for customized or programmable operation to control the tool and accessory combinations. The programmable configurations may be accessed for individual users (e.g., physicians, surgeons, technicians, etc.) to customize the operation of the control console. In order to support the complex operation of the system, a user interface may be incorporated via a display device of the control console. In operation, the user interface of the control console may depict various representative identification symbols identifying the interchangeable accessory or accessories in use. The identification symbol for each of the interchangeable accessories may be displayed on the display device near an interface or communication port of the corresponding surgical tool. In this way, the control console may configure the user interface to depict the identification symbol for the interchangeable accessory in an intuitive way.

Each of the interchangeable accessories may be identified by the controller based on communication from an identification device via a communication circuit. The communication between the communication circuit and the identification device may be wireless or may be implemented by various conductive connections. A control circuit of the control console may access the identification information from the identification device of the accessory and utilize the identification information to identify and display an identification symbol for the interchangeable accessory. The identification symbol may correspond to a color-coded graphic or depiction of the interchangeable accessory. The identification information may further include dimensions (e.g., length, diameter, travel distance, etc.), materials or design variations (e.g., curved, angled, etc.), operating ranges (e.g., maximum speed, oscillation rate or count, feed rate, plunge rate, etc.), and various other settings that may relate to the operation of the interchangeable accessory.

In various implementations, the disclosure further provides for one or more of the following features alone or in combination:
a control console for at least one surgical device is provided including at least one drive circuit configured to control with the surgical device via at least one interface port; a display device configured to display operating state information of the surgical device; a communication circuit configured to communicate with an identification device of an interchangeable accessory of the surgical device; and a control circuit configured to: access identification information of the interchangeable accessory from the identification device via the communication circuit; and display the identification information on the display device, wherein the identification information comprises an identification symbol of the accessory;
the identification symbol comprises a graphic identifying the interchangeable accessory;
the identification symbol comprises a color coded identification card;
the identification device comprises a wireless transmitter configured to communicate the identification information to the communication circuit from the interchangeable accessory;
the controller is configured to control an operating state of the drive circuit of the surgical device via the interface port in response to the identification information;
the identification symbol is received from the identification device of the accessory;
the identification symbol comprises at least one of a depiction of the accessory, a dimension of the accessory, and an operating range of the accessory;
the at least one control circuit is configured to control a plurality of surgical devices comprising a first device and a second device;
the first device comprises a first identification device and the second device comprises a second identification device, and wherein the controller is configured to access first identification information of a first accessory in connection with the first device from a first identification device via the communication circuit and access second identification information of second accessory in connection with the second device from a second identification device via the communication circuit;
the control circuit is further configured to control the display device to display device to display a first identification symbol of the first accessory in response to the connection of the first device to the control console and to display a second identification symbol of the second accessory in response to the connection of the second device to the control console;
the control circuit is further configured to independently control operating states of each of the first surgical device and the second surgical device via the at least one interface port in response to the first identification information and second identification information;
the first identification information comprises a first operating configuration for the first surgical device in coordination with the first accessory, and the second identification information comprises a second operating configuration for the second surgical device in coordination with the second accessory;
the at least one interface port comprises a first port located on the control console adjacent to a first side portion of the display device and a second port located on the control console adjacent to a second side portion of the display device, wherein the controller is further configured to control the display device to display a first identification symbol of the first accessory on the first side portion, and a second identification symbol of the second accessory on the second side portion; and/or
the accessory is an interchangeable cutting tools for surgical operations comprising at least one of a shaver, a rasp, a burr, a dissector, a drill, a saber, a resector, and a blade.

In some aspects, the disclosure provides for a method for controlling a surgical device including accessing identification information from an identification device of at least one interchangeable accessory. The method further includes configuring an operating configuration of at least one surgical device in response to the identification information. The control instruction is communicated for an actuator of the at least one surgical device via a first port of a plurality of ports of a connection interface. The first port of the connection interface is distinguished from the plurality of ports of the connection interface by displaying an identification symbol identifying the interchangeable accessory on a display screen.

In various implementations, the disclosure further provides for one or more of the following features or steps alone or in combination:
the identification symbol is received from the identification device of the interchangeable accessory;
the identification symbol is accessed as a picture stored in the identification device of an interchangeable accessory of the surgical device;
providing the at least one interchangeable accessory in packaging that bears the identification symbol stored in the identifying information;
the at least one surgical device comprises the first device in connection with a first interchangeable accessory via the first port and a second device in connection with a second interchangeable accessory via a second port of the plurality of ports;
distinguishing the first port in connection with the first device from a second port of the plurality of ports in communication with the second device by: displaying a first identification symbol identifying the first interchangeable accessory on a first side portion of the display screen adjacent to the first port; and displaying a second identification symbol of the second interchangeable accessory of the second device on a second side portion of the display screen adjacent to the second port;
the identification information is applied configuring the operating configuration of at least one surgical device in response to the at least one surgical device in connection with the at least one interchangeable accessory and the control console;
accessing a user profile from a user database; and setting a control configuration of the at least one surgical device in combination with the at least one interchangeable accessory in response to a user setting of the user profile;
the user setting of the user profile comprises an input configuration of a user interface of the at least one surgical device; and/or
the user setting comprises a user-defined operating range for the at least one surgical device.

In yet another example, the disclosure provides for a control console for a surgical device comprising a display device comprising a first side portion and a second side portion. A communication interface includes a first port disposed adjacent to the first side portion and a second port disposed adjacent to the second side portion. The first port is configured to control a first surgical device and the second port is configured to control a second surgical device. At least one communication circuit is in communication with a first identification circuit of a first interchangeable accessory of the first surgical device and a second identification circuit of a second interchangeable accessory of the second surgical device. The first identification circuit comprises first identifying information of the first interchangeable accessory and the second identification circuit comprises second identifying information of the second interchangeable accessory. A control circuit is configured to access the first identification information and the second identification information via the at least one communication circuit. The controller further controls a first identifying symbol to be displayed on the display device based on the first identification information and a second identifying symbol to be displayed on the display device based on the second identification information. The first identifying symbol and the second identifying symbol may be accessed in the first identifying information and the second identifying information.

These and other features, objects and advantages of the present disclosure will become apparent upon reading the following description thereof together with reference to the accompanying drawings.

### BRIEF DESCRIPTION OF THE DRAWINGS

FIG. 1 is a diagram demonstrating a system comprising a control console configured to operate a plurality of surgical devices and interchangeable accessories;

FIG. 2 is a schematic diagram of a control console interfaced with an exemplary surgical device;

FIG. 3 is a diagram demonstrating display device and user interface of a control console;

FIG. 4A is a diagram demonstrating a graphic depiction of a user interface of a control console providing user-specific settings for interchangeable accessories;

FIG. 4B is a diagram demonstrating a graphic depiction of a user interface of a control console providing user-specific settings preferences for handpiece configurations;

FIG. 4C is a diagram demonstrating a graphic depiction of a user interface of a control console providing user-specific settings for peripheral controls or pedal configurations;

FIG. 5 is a flow chart demonstrating an exemplary method for controlling and operating a control console; and

FIG. 6 is a block diagram demonstrating a control system in connection with a surgical device and input device in accordance with the disclosure.

### DETAILED DESCRIPTION OF THE PREFERRED EMBODIMENT

The disclosure generally provides for systems and methods related to the control and adjustment of operating configurations for a variety of surgical devices. Due to the variety of compatible surgical devices, the control console may be required to access and apply diverse operating routines and configurations to provide effective control. Each of the surgical devices controlled by the control console may further be compatible with a variety of interchangeable accessories. The accessories may have specific operating ranges (e.g., operating speeds, oscillation rates, etc.) and operating features (e.g., operating direction, oscillation capability, plunging or extension range, etc.) that may be identified by the control console to provide corresponding control schemes. As provided in the following detailed examples, the disclosure may provide for improved hardware configurations and methods for operating highly configurable devices in an intuitive manner.

In some cases, systems may provide for selective illumination of one or more indicator lights to identify the connectivity of the ports with compatible devices. However, due to the complex nature of interchangeable accessories implemented in the disclosed system, illumination or indications may be inadequate to distinguish among the wide variety of compatible devices. The control console may include a display device configured to display one or more identifying graphics that visually communicate a configuration of a connected accessory for each connected surgical tool via a representative symbol. The identification symbols may be displayed proximate or adjacent to an interface port associated with the connected surgical device. In this way, the control console may provide for an intuitive user interface that allows operators to easily distinguish among surgical tools and connected interchangeable accessories.

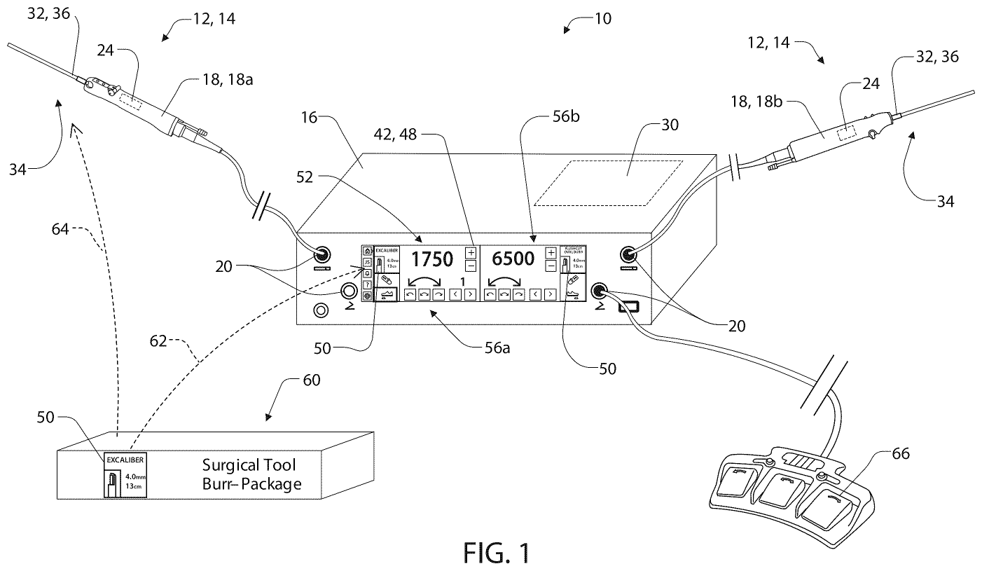

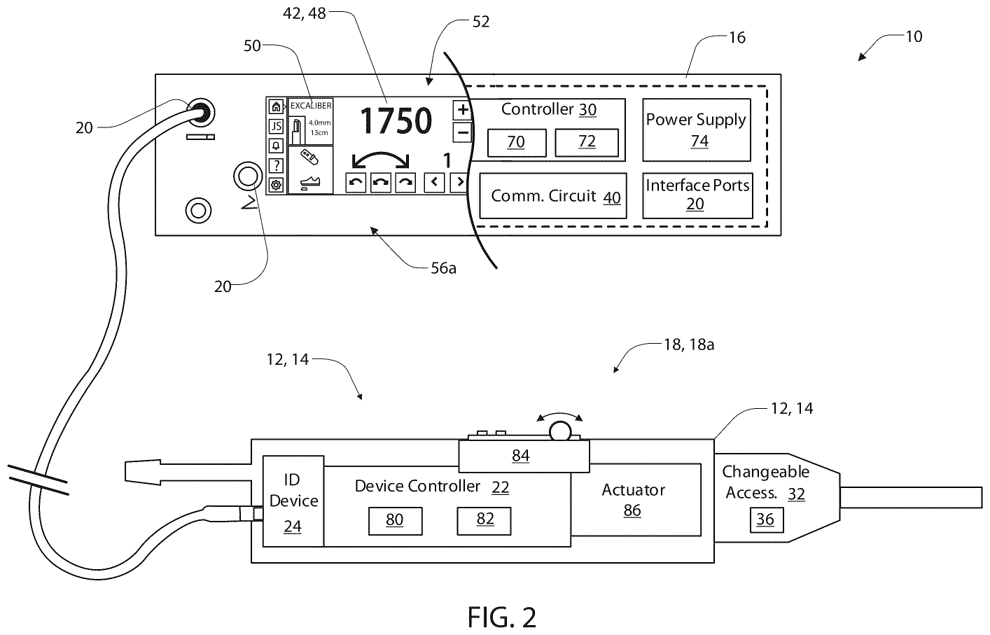

Referring now to FIGS. 1 and 2, exemplary diagrams of a control system 10 for a surgical device 12 are shown. As described, the surgical devices 12 may correspond to tools, input control devices, peripherals, or related accessories. For clarity, the depicted surgical device 12 is exemplified in the form of a hand-held instrument or handpiece 14, which is powered by a control console 16. In operation, the control console 16 may supply operating power and control signals to multiple handpieces 14, each of which may have a different operating configuration 18 (e.g., first configuration 18a, second configuration 18b). Each of the surgical devices 12 may connect to the control console 16 via an interface port 20 configured to communicate operating power and control signals to device controllers 22 of the surgical devices 12. In this way, control console 16 may provide for concurrent and independent control of the plurality of surgical devices 12 via the interface ports 20.

To identify the operating configuration 18 associated with each of the surgical devices 12, the system 10 may receive an identification signal from an identification device 24. In operation, upon connecting each of the surgical devices 12 to the interface ports 20, operating energy may be supplied to the surgical device 12 to activate the identification device 24. Once activated, the identification device 24 may communicate a signal (e.g., voltage, digital signal, etc.) to a controller 30 of the control console 16 that identifies the operating configuration 18 of the connected surgical device 12. In response to the identification signal from each of the surgical devices 12, the controller 30 may identify the operating configuration 18 and assign a corresponding control configuration to the associated interface port 20. In this way, the controller 30 of the control console 16 may identify and distinguish the operating configurations 18 of a wide variety of surgical devices 12 to effectively control and/or communicate with such devices.

As previously discussed, each of the surgical devices 12 may drive or control the motion of a plurality of interchangeable accessories 32. For example, the interchangeable accessories 32 compatible with each configuration 18 of the surgical devices 12 may include a variety of cutting tools or surgical tools including, but not limited to, a shaver, a rasp, a burr, a dissector, a drill, a saber, a resector, a blade, etc. In addition to the operating configurations 18 of each of the surgical devices 12, the interchangeable accessories 32 may further include accessory-specific control configurations 34. In order to identify the associated control configuration 34, the interchangeable accessories 32 may include an identification circuit 36. The identification circuit 36 may be configured to communicate identification data or information identifying the control configuration 34 of the interchangeable accessory 32 to a communication circuit 40 (see, FIG. 2) of the control console 16. In various implementations, the identification circuits 36 may communicate identification information to the communication circuit 40 indicating an operating configuration, operating range, and/or operating features of each interchangeable accessory 32. In some examples, the identification circuit 36 may communicate the identification information for the interchangeable accessory 32 via one or more signals conducted to the communication circuit 40 via the surgical device 12 and interface ports 20. Additionally, in some cases, the identification circuit 36 may wirelessly communicate the identification information to the communication circuit 40. Accordingly, the system 10 may be flexibly implemented to suit a variety of applications.

In cases where the identification information is communicated wirelessly, the identification circuit 36 of the interchangeable accessory 32 may correspond to a wireless transmitter (e.g., radio frequency identification [RFID] device) and the communication circuit 40 may correspond to a wireless receiver or transceiver. In these cases, the controller 30 may identify the operating configuration 18 of the surgical devices 12 via the identification device 24 in communication with the interface port 20. Additionally, the controller 30 may further identify the control configuration 34 associated with the interchangeable accessory 32 via the communication circuit 40. Once the devices 12 and associated interchangeable accessories 32 are connected to the control console 16, the controller 30 may utilize the operating configuration 18 of the handpiece 14 and the control configuration 34 of the accessory 32 to adjust and control the operation of each of the surgical devices 12.

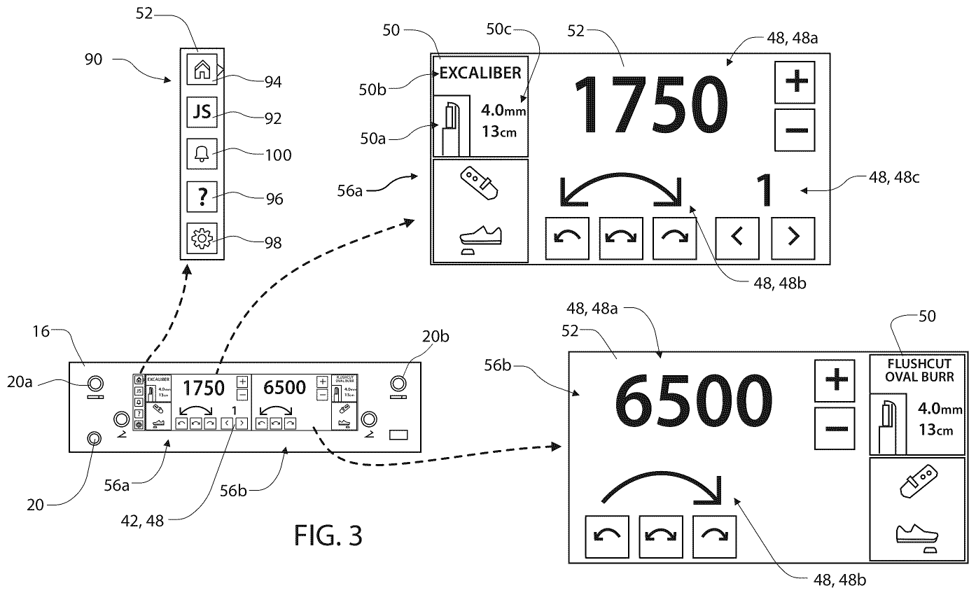

While the controller 30 may automatically assign configurations and maintain them throughout connection and operation, distinguishing among multiple connected surgical devices 12 may be challenging for an operator. Accordingly, the control console 16 may include a display device 42 configured to display the operating state or control information 48 for each of the surgical devices 12. In addition to the operating information, an identification symbol 50, graphic and/or color-coded identifier, may be displayed and associated with the connected interchangeable accessory 32. As shown in FIG. 1 and discussed later in further detail in reference to FIG. 3, the display device 42 demonstrates a user interface 52 that generally includes a first side portion 56a and a second side portion 56b, which may be positioned on opposing sides of the display device 42. The first and second side portions 56 may provide for a divided or split-screen configuration of the user interface 52. As shown, the control information 48 for each of the surgical devices 12 may include an operating speed 48a, mode 48b, number of rotations per cycle 48c, and various additional information associated with the operation of the surgical device 12 (see, FIG. 3). The identification symbol 50 may demonstrate various information related to the interchangeable accessory 32, which may include a graphic representation of the accessory, dimensional information, an identification name or tool type, and various additional information associated with the interchangeable accessory 32.

The split-screen configuration of the user interface 52 demonstrated on the display device 42 may provide an intuitive identification of the interchangeable accessory 32 by displaying the identification symbol 50 adjacent to or on a corresponding side 56a or 56b as the associated interface port 20 connected to the surgical device 12. In addition to the readily identifiable position of the identification symbol 50 adjacent the corresponding interface port 20, the identification symbol 50 may also be depicted on packaging 60 (e.g., wholesale or retail packaging) for each of the interchangeable accessories 32. As demonstrated by arrow 62 shown in FIG. 1, the identification symbol 50 may be demonstrated in a similar color and format on the packaging 60 as well as the display 42. Accordingly, when the interchangeable accessory 32 is installed on the surgical device 12, as represented by arrow 64, the user may begin to associate the identification symbol 50 with the surgical device 12 and interface port 20. In this way, the identification symbol 50 may be associated by the user with the operation of the interchangeable accessory 32 during a setup procedure, which may assist in the recognition of the identification symbol 50 on the display 42 during operation.

As discussed herein, the term “adjacent” may refer to an object that is located relative to something without or absent an intervening object of the same kind in between. More generally, adjacent may refer to a proximity to another object. In reference to the position of the ports 20 relative to the display 42, the distance between these features may vary depending on the proportions of the display 42 and the ports 20 of the connection interface of the control console 16. The relationship between the identification symbol 50 and the associated port 20 may be significantly free of intervening ports or like elements, such that the identification symbol 50 demonstrated on the screen 42 is readily associated with the corresponding interface port 20.

In addition to the control of the surgical devices 12, the control console 16 may further communicatively connect with a variety of peripheral devices or accessories 66 (e.g., foot switches, pedals, remote controls, etc.). The peripheral devices 66 may similarly be connected to the interface ports 20 of the control console 16 and configured via the identification device 24 or identification circuit previously described. In some examples, the peripheral devices 66 may correspond to input or control devices that may receive inputs to control the handpieces 14. Accordingly, the system 10 may be implemented to control various surgical devices or handpieces 14 based on input signals supplied to the controller 30 via a wide variety of controls or peripheral devices 66.

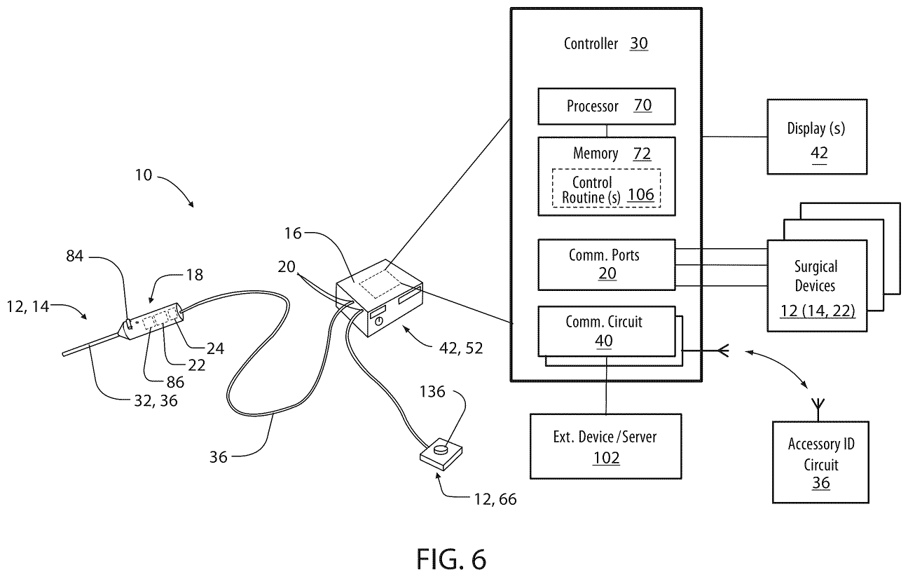

Referring now to FIG. 2 in further detail, the control console 16 is shown connected to an exemplary handpiece 14 via the interface port 20. As shown, the controller 30 of the control console 16 may comprise a processor 70. The processor 70 may be operable to access information related to the operating configurations 18 or control configurations 34 of the surgical devices 12 and the interchangeable accessories 32 via one or more data sets or tables that may be stored in a memory 72. Additionally, the controller 30 of the system 10 may be in communication with one or more servers or external devices via the communication circuit 40. A power supply 74 may supply operating energy to one or more drive circuits, which may include a plurality of switching devices configured to supply control signals to each of the surgical devices 12 or handpieces 14. Further detailed discussion regarding the hardware configuration (e.g., processors, circuits, memory, communication devices, etc.) of the controller 30 and further supporting details of the technical operation of the system 10 are provided in reference to FIG. 6.

Though the operating configurations and corresponding applications of each of the surgical devices 12 or handpieces 14 may vary widely, FIG. 2 demonstrates an exemplary configuration for clarity. As previously discussed, the surgical device 12 may include a device controller 22 that may interpret and exchange control instructions with the controller 30 of the control console 16. The device controller 22 may include a processor 80 in communication with a memory 82, which together may be configured to communicate and interpret operating signals to the controller 30 of the control console 16 via the interface ports 20. In this configuration, inputs received via a user interface 84 of each of the surgical devices 12 may be communicated from the device controller 22 to the controller 30 of the control console 16 to adjust and control the operation of the handpiece 14.

The device controller 22 may further be in communication with an actuator 86, which may correspond to an electric motor and drive mechanism. The actuator 86 may generate rotational motion and/or translation of motion to control the operation of the interchangeable accessory 32. Though not shown in detail, the interchangeable accessory 32 may engage the actuator 86 via a coupler or chuck, such that the rotational or translation of motion of the actuator 86 may result in the corresponding controlled movement of the interchangeable accessory 32. As previously discussed, the interchangeable accessories 32 may correspond to reciprocating or rotating cutting or oscillating blades that may be implemented in surgery for surface preparations, resections, and/or various cutting or burring operations. For example, the interchangeable accessories 32 may be configured to provide various cutting, drilling, burring, or cutting application for surgical operations.

Though discussed primarily in reference to the handpieces 14, the surgical devices 12 may also correspond to a variety of medical devices (e.g., lasers, radio frequency ablation devices, pumps, vacuums/suction devices, imagers, etc.). The surgical devices 12 may similarly be interfaced with the control console 16 based on the corresponding device information accessed from the identification device 24 and identification circuit 36. Though discussed in reference to specific examples, the identification device 24 of the surgical device 12 and the identification circuit 36 of the interchangeable accessory 32 may be implemented in a variety of ways as discreet devices or incorporated in the structure of related devices (e.g., the device controller 22). More generally, each of the computerized or electronic devices may be implemented based on a wide variety of technologies and structures that are not limited to the specific examples provided. For example, each of the processors, controllers, or other control units described herein may be implemented via one or more microprocessors, microcontrollers, application-specific integrated controllers (ASIC), or other circuitry configured to perform instructions, computations, and control various input/output signals that may provide for the operation of the system 10 as discussed herein. Accordingly, variations to the specific data processing structures and corresponding circuits described herein will be appreciated by those skilled in the art to be embodied by the general teachings of this disclosure.

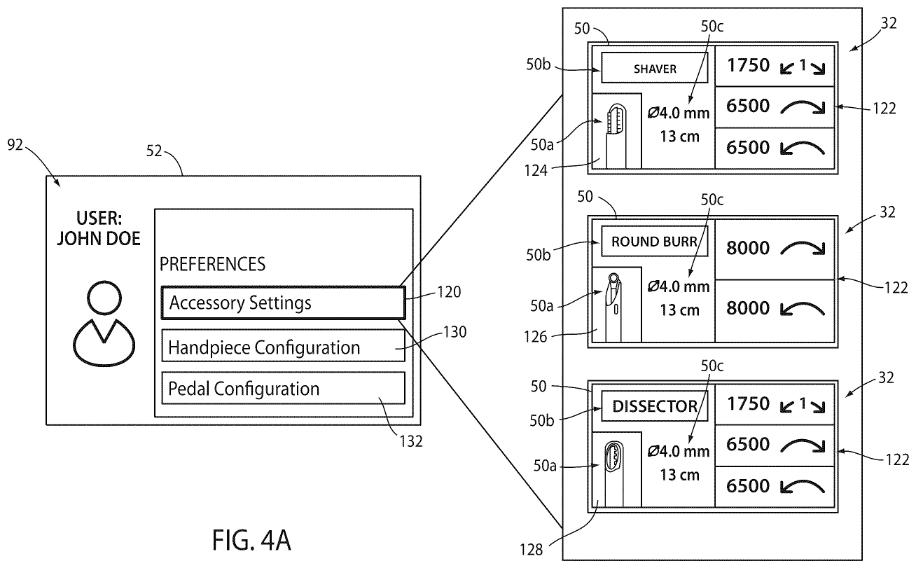

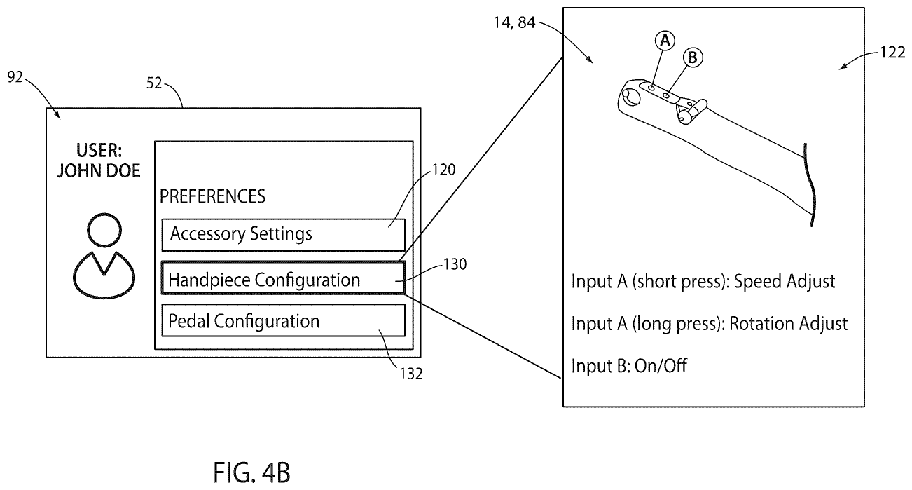

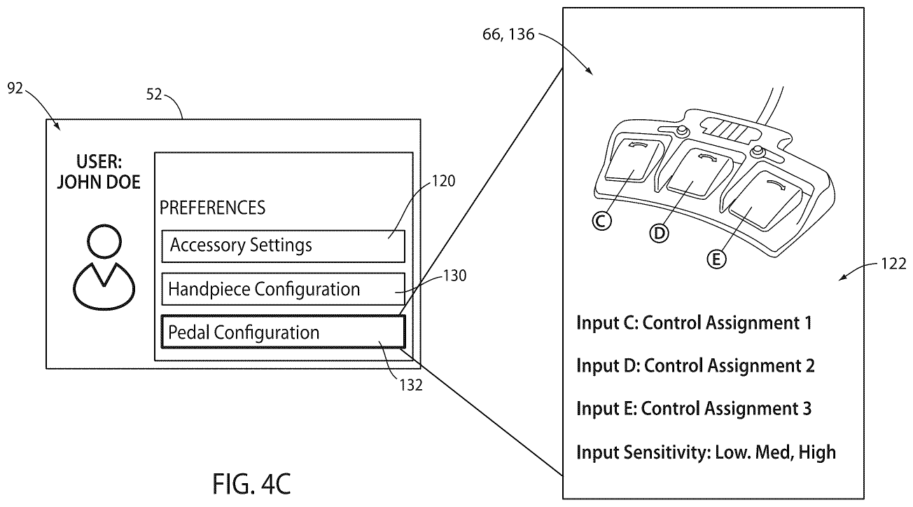

Referring now to FIG. 3, the user interface 52 of the exemplary control console 16 is shown in further detail. The first side portion 56a is shown separate from second side portion 56b for clarity. However, the portions 56a, 56b may correspond to flexibly configured regions of a contiguous display panel. An operating menu 90 is shown that provides for access to one or more user profiles 92, as well as navigation to a home screen 94, help menu 96, settings menu 98, and alerts menu 100. As discussed later in reference to FIG. 6, the user profile 92 may be accessed by the controller 30 via a user database, which may be stored in an external device or server 102 (see, FIG. 6). The user profile 92 may provide for an associated user to store specific user-defined configurations including, but not limited to, operating preferences (e.g., speed ranges, button configurations, shortcuts, etc.) for the operation of each combination of the interchangeable accessories 32 and surgical devices 12 or handpieces 14 discussed herein. Such preferences are further discussed in reference to FIGS. 4A, 4B, and 4C. The corresponding settings of the control console 16 may be accessed via the settings menu 98 and alerts or notifications to the control console 16 may be accessed via the alerts menu 100.

As previously discussed, the first side portion 56a of the user interface 52 may depict an identification symbol 50 associated with the adjacent interface port 20—referred to as a first interface port 20a for clarity. Similarly, a second interface port 20b may be located adjacent the second side portion 56b depicting the identification symbol 50 corresponding to the interchangeable accessory 32 of the surgical device 12 connected to the corresponding port 20b. In this configuration, the user interface 52 may present an operator of the control console 16 with the identification symbols 50 in such a way that the user may readily associate the operating control information 48, including the speed 48a, mode 48b, number of rotations 48c, etc. of the surgical device 12 with the corresponding interface port 20a, 20b. Each of the identification symbols 50 may include detailed information identifying the corresponding interchangeable accessory 32. For example, each of the identification symbols 50 may include a representative diagram or graphic 50a depicting the interchangeable accessory 32. Additionally, the identification symbol 50 may include a name or designation 50b (e.g., a description, title, or class of the device) as well as dimensional information 50c (e.g., length, diameter, etc.) of the interchangeable accessory 32. The identification symbols 50 may be color-coded with a variety of readily distinguishable colors to further differentiate the interchangeable accessories 32 in connection with the surgical devices 12. As shown, a first color 108 may be used to designate the interchangeable accessory 32 or first accessory shown on the first side portion 56a and a second color 110 may be implemented to designate the interchangeable accessory 32 associated with the second side portion 56b. In this way, the user interface 52 may provide for an intuitive method of distinguishing among the extensive variety of interchangeable accessories 32 to ensure that the correspond operation and control settings of the control console 16 are readily distinguishable.

Referring now to FIGS. 4A, 4B, and 4C, an exemplary interface is shown demonstrating various preferences associated with the user profile 92. As shown in FIG. 4A, preferences may be assigned in relation to each of a plurality of the interchangeable accessories 32 as accessory settings 120. For example, user-specified control information 122 is shown for a first accessory 32a, a second accessory 32b, and a third accessory 32c. The interchangeable accessories 32a, 32b, and 32c are color-coded in corresponding identifying colors, including a first color 124, a second color 126, and a third color 128. Accordingly, the identification symbols 50 may be demonstrated including the graphic 50a, designation 50b, dimensional information 50c, and/or the color-coding in the accessory settings or preferences page to further ensure that the corresponding accessories 32 are readily identifiable without confusion.

Referring now to FIGS. 4B and 4C, the preferences associated with the user profile 92 may further include a handpiece configuration 130 and a pedal configuration 132. The handpiece configuration 130 and the pedal configuration 132 may each provide for user-specific programming. The programming may vary depending on the complexity of the device 12 and may include custom configurations for a plurality of user inputs of the user interface 84 and/or an interface configuration 136 of the peripheral device 66, shown as a pedal control 138. In the example, each of the inputs (e.g., a, b, c, d, e) may be adjusted and assigned by the controller 30 to control different operations of surgical devices or provide user-specific operation and sensitivity settings. For example, in reference to the operation of the handpiece 14, Input A is associated with a speed adjustment upon receiving a short press and may provide for a directional change in response to a long press. Input B may provide for the activation or deactivation of the handpiece 14.

In reference to FIG. 4C, control assignments 1, 2 and 3 may be associated to provide specific controls (e.g., forward, reverse, oscillating, etc.) in reference to each of the Inputs C, D, and E. Additionally, the preferences associated with each user profile 92 may provide for the adjustment of sensitivity settings for each of the inputs ranging from a low sensitivity to a high sensitivity or over a corresponding numeric range (e.g., relative sensitivities 1-10). Accordingly, by accessing a user profile 92, the operation of each of the surgical devices 12 and corresponding interchangeable accessories 32 may be customized and associated with user-specified control information 122.

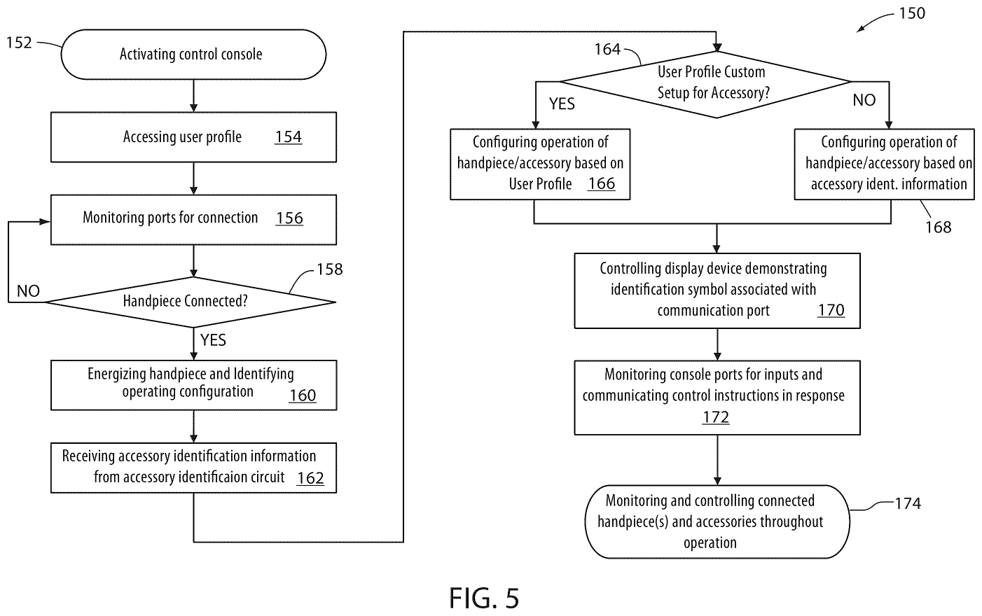

Referring now to FIG. 5, a flow chart is shown demonstrating a method 150 for operating the control console 16. Beginning at step 152, the method 150 may begin in response to the activation of the control console 16. Once activated, the controller 30 may access the user profile 92 (154), which may provide for various user-specified control information 122 associated with each user. Throughout operation, the controller 30 may monitor the interface ports 20 for the connection of a surgical device 12 or handpiece 14 (156). If the handpiece 14 is identified as being connected in step 158, the method 150 may proceed to step 160 to begin communication with the handpiece 14. If the handpiece 14 is not identified as being connected in step 158, the method 150 may return to step 156 to monitor for connected devices 12.

In order to identify the operating configuration of the handpiece 14 in step 160, the controller may energize the identification device 24 to initiate communication. Additionally, the communication circuit 40 may receive identification information for the interchangeable accessory 32 from the identification circuit 36 (162). In response to the control configuration 34 of the interchangeable accessory 32 and the operating configuration 18 of the handpiece 14, the controller 30 may continue to step 164. In step 164, the controller 30 may configure the operation of the handpiece 14 to control the interchangeable accessory 32 based on the user profile 92 and corresponding custom settings or control configurations 34. If the custom user profile 92 is associated with the user, the method 150 may continue to step 166 to configure the operation based on the user profile 92. If a user profile 92 is not accessed or preferences have not been set, the method 150 may continue to step 168 to configure the operation of the handpiece 14 and interchangeable accessory 32 based on factory or default settings. In this way, the method 150 may provide for a custom or predefined configuration for the operation of various handpieces 14 in coordination with a variety of interchangeable accessories 32.

Once the handpiece 14 and interchangeable accessory 32 are configured for operation with the controller 30, the method 150 may continue to control the display device 42 to demonstrate a corresponding identification symbol 50 demonstrating the interchangeable accessory 32 adjacent to or proximate the corresponding interface port 20 (170). Following step 170, the controller 30 may monitor inputs associated with the user interface 84 of the handpiece 14 or inputs by peripheral devices 66 and communicate control instructions to the handpiece 14 in response to the inputs (172). Following step 172, the method 150 may continue to monitor and control the connected handpieces 14 or surgical devices 12 throughout the operation of the control console 16 (174). Accordingly, the system 10 may provide for an intuitive method to quickly identify and configure the control console 16 for operation with a wide variety of surgical devices 12 or handpieces 14 associated with various interchangeable accessories 32.

Referring now to FIG. 6, the control system 10 is shown demonstrating the control console 16 and further details of the console controller 30. In operation, the controller 30 may receive inputs via one or more user interfaces 84, 136 of the surgical devices 12 and/or peripheral devices 66. In various examples, the operation of the system 10 is discussed in reference to the operation of the handpiece 14. However, it shall be understood that the operation of the system 10 may commonly provide for the concurrent use and control of two or more surgical devices 12, which may include various handpieces 14, peripheral devices 66, remote controls, and various other devices or accessories that may be beneficial in a medical or surgical environment. For example, the surgical devices 12 may include various laser or radio frequency cutting or operating utilities in the form of ablation devices, catheters, pumps, suction or aspiration devices, and similar tools that may also correspond to handpieces 14.

In some implementations, the system 10 may include one or more display devices 42 that communicate with various controllers and surgical devices 12 similar to those discussed herein. In some examples, the surgical device 12 may correspond to one or more imaging devices, which may be connected to or in communication with the control console 16. The image data captured by such surgical devices 12 may be presented by one or more communicatively connected display devices (e.g., display monitors). The image data as well as the identification symbol 50 and control information 48 may be displayed on such display devices to further promote ease of identifying and tracking the configuration of each of the connected devices 12. For example, one or more displays connected to the system 10 may display or report the identification symbol 50 or similar control information 48, including the speed 48a, mode 48b, number of rotations 48c, etc. for the surgical devices 12. The accessory information of the interchangeable accessories 32 as well as the operating configurations of the surgical devices 12 may also be reported to or communicated to the external device or server 102 (e.g., remote peripherals, servers, communication networks, etc.) in communication with the control console 16 via the communication or interface ports 20, communication circuits 40, or various communication interfaces.

As previously discussed, the control console 16 may be in communication with one or more peripheral devices 66 or accessories associated with the operation of the control console 16. For example, the peripheral devices 66 may correspond to one or more electronic or electromechanical buttons, triggers, or pedals (e.g., pressure sensitive or single actuation foot pedals), and additional devices communicatively connected to the interface ports 20. The display 42/user interface 52 of the control console 16 may include one or more switches, buttons, dials, and/or displays, which may include soft-key or touchscreen devices incorporated in a display (e.g., liquid crystal display [LCD], light emitting diode [LED] display, cathode ray tube [CRT], etc.). In response to inputs received from the display 42/user interface 52, the controller 30 may activate or adjust the settings of the control signals communicated to the surgical devices 12. The control signals generated by the console controller 30 may be configured for operation in response to the operating configuration 18 identified from the identification device 24 as well as the accessory identification circuit 36. The output signals communicated from the ports 20 to the surgical devices 12 or handpieces 14 may be generated by various signal generators, motor controllers, or power supplies that may provide for operation of power electronic operations (e.g., motor drive signals and supply current), which may be controlled and configured for operation based on the instructions, commands, or signals communicated from the processor 80 of the console controller 30 for the associated operating configuration 18. Accordingly, the console controller 30 may be operable to generate signals to drive or control the motion, rotation, activation, intensity, and various other operating characteristics of the surgical devices 12.

The performance or specifications of the control console 16 (e.g., rating of power supply 74, heat dissipation, etc.) may be designed to accommodate the target properties of the control signals for each of the surgical devices 12 and peripheral devices 66 associated with the system 10. The processor 70 of the controller 30 or, more generally, the processors 70, 80 discussed herein may be implemented as microprocessors, microcontrollers, application-specific integrated circuits (ASIC), or other circuitry configured to perform instructions, computations, and control various input/output signals to control the control system 10. The instructions and/or control routines 180 of the system 10 may be accessed by the processors 70, 80 via a memory 72, 82. The memory 72, 82 may comprise random access memory (RAM), read only memory (ROM), flash memory, hard disk storage, solid state drive memory, etc. Each of the processors 70, 80 and memory devices 72, 82 may be implemented to suit the corresponding functionality or sophistication of the surgical devices 12 and the corresponding control requirements of the controller 30.

The controller 30 may incorporate additional communication circuits or input/output circuitry, generally represented in FIG. 6 as the communication circuit(s) 40, which may be implemented to communicate with one or more peripherals, devices, remote computers or servers, etc. The communication circuit(s) 40 may complement or support the operating capability of the interface ports 20. In general, the communication circuit 40 may provide for communication via a variety of communication protocols to support operation of the surgical devices 12. In an exemplary embodiment, the circuitry associated with the interface ports 20 may include digital-to-analog converters, analog-to-digital converters, digital inputs and outputs, as well as one or more communication interfaces or buses. The circuits associated with the interface ports 20 and/or the communication circuit 40 may be implemented with various communication protocols, such as serial communication (e.g., CAN bus, I2C, etc.), parallel communication, or network communication (e.g., RS232, RS485, Ethernet). In some cases, the communication circuit 40 may also provide for wireless network communication (Wi-Fi, Bluetooth®, Ultra-wideband [UWB], etc.). In some examples, the controller 30 may be in communication with one or more of the external devices 102 (e.g., control devices, peripherals, servers, etc.) via the communication circuit 40. Accordingly, the control console 16 may provide for communication with various devices to update, maintain, and control the operation of the system 10.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

The claims:

1. A control console for at least one surgical device comprising:
at least one drive circuit configured to control the surgical device via at least one interface port;
a display device configured to display operating state information of the surgical device;
a communication circuit configured to communicate with an identification device of an interchangeable accessory of the surgical device; and
a control circuit configured to:
access identification information of the interchangeable accessory from the identification device via the communication circuit; and
display the identification information on the display device, wherein the identification information comprises an identification symbol of the accessory.

2. The control console according to claim 1, wherein the identification symbol comprises a graphic identifying the interchangeable accessory.

3. The control console according to claim 1, wherein the identification symbol comprises a color-coded identification card.

4. The control console according to claim 1, wherein the identification device comprises a wireless transmitter configured to communicate the identification information to the communication circuit from the interchangeable accessory.

5. The control console according to claim 1, wherein the controller is further configured to:
control an operating configuration of the drive circuit of the surgical device via the interface port in response to the identification information.

6. The control console according to claim 1, wherein the identification symbol is received from the identification device of the accessory.

7. The control console according to claim 1, wherein the identification symbol comprises at least one of a depiction of the accessory, a dimension of the accessory, and an operating range of the accessory.

8. The control console according to claim 1, wherein the at least one control circuit is configured to control a plurality of surgical devices comprising a first device and a second device.

9. The control console according to claim 1, wherein the first device comprises a first identification device and the second device comprises a second identification device, and wherein the controller is configured to:
access first identification information of a first accessory in connection with the first device from a first identification device via the communication circuit; and
access second identification information of second accessory in connection with the second device from a second identification device via the communication circuit.

10. The control console according to claim 9, wherein the control circuit is further configured to:
control the display device to display a first identification symbol of the first accessory in response to the connection of the first device to the control console and to display a second identification symbol of the second accessory in response to the connection of the second device to the control console.

11. The control console according to claim 9, wherein the control circuit is further configured to:
independently control operating states of each of the first surgical device and the second surgical device via the at least one interface port in response to the first identification information and second identification information.

12. The control console according to claim 11, wherein the first identification information comprises a first operating configuration for the first surgical device in coordination with the first accessory, and the second identification information comprises a second operating configuration for the second surgical device in coordination with the second accessory.

13. The control console according to claim 9, wherein the at least one interface port comprises a first port located on the control console adjacent to a first side portion of the display device and a second port located on the control console adjacent to a second side portion of the display device, wherein the controller is further configured to control the display device to display a first identification symbol of the first accessory on the first side portion, and a second identification symbol of the second accessory on the second side portion.

14. The control console according to claim 9, wherein the accessory is an interchangeable cutting tool for surgical operations comprising at least one of a shaver, a rasp, a burr, a dissector, a drill, a saber, a resector, and a blade.

15. A method for controlling a surgical device comprising:
accessing identification information from an identification device of at least one interchangeable accessory;
controlling an operating configuration of at least one surgical device in connection with the interchangeable accessory in response to the identification information;
communicating a control instruction for an actuator of the at least one surgical device via a first port of a plurality of ports of a connection interface; and
distinguishing the first port of the connection interface from the plurality of ports of the connection interface, wherein the first port is distinguished by displaying an identification symbol identifying the interchangeable accessory on a display screen.

16. The method according to claim 15, wherein the identification symbol is received from the identification device of the interchangeable accessory.

17. The method according to claim 15, wherein the identification symbol is accessed as a picture stored in the identification device of an interchangeable accessory of the surgical device.

18. The method according to claim 15, wherein the at least one surgical device comprises the first device in connection with a first interchangeable accessory via the first port and a second device in connection with a second interchangeable accessory via a second port of the plurality of ports; and
the method further comprising:
distinguishing the first port in connection with the first device from a second port of the plurality of ports in communication with the second device by:
displaying a first identification symbol identifying the first interchangeable accessory on a first side portion of the display screen adjacent to the first port; and
displaying a second identification symbol of the second interchangeable accessory of the second device on a second side portion of the display screen adjacent to the second port.

19. A control console for a surgical device comprising:
a display device comprising a first side portion and a second side portion;
a communication interface comprising a first port disposed adjacent to the first side portion and a second port disposed adjacent to the second side portion, wherein the first port is configured to control a first surgical device and the second port is configured to control a second surgical device;
at least one communication circuit in communication with a first identification circuit of a first interchangeable accessory of the first surgical device and a second identification circuit of a second interchangeable accessory of the second surgical device, wherein the first identification circuit comprises first identifying information of the first interchangeable accessory and the second identification circuit comprises second identifying information of the second interchangeable accessory; and
a control circuit configured to:
access the first identification information and the second identification information via the at least one communication circuit;
display a first identifying symbol on the first side portion of the display device based on the first identification information; and
display a second identifying symbol on the second side portion of the display device based on the first identification information.

20. The control console according to claim 19, wherein the first identifying symbol is accessed in the first identifying information and the second identifying symbol is accessed in the second identifying information.
